(19) United States
(12) Patent Application Publication
(10) Pub. No.: US 2005/0080422 A1
(43) Pub. Date: Apr. 14, 2005

(54) Instruments for use with implants, and methods

(75) Inventors: John F. Otte, St. Anthony, MN (US); James R. Mujwid, Crystal, MN (US); Ishmael Bentley, Eagan, MN (US)

Correspondence Address: MERCHANT & GOULD PC, P.O. BOX 2903, MINNEAPOLIS, MN, 55402-0903, US

(73) Assignee: Centerpulse Spine-Tech, Inc.

(21) Appl. No.: 10/685,768

(22) Filed: Oct. 14, 2003

(51) Int. Cl. A61B 17/00

(52) U.S. Cl. 606/85

(57) ABSTRACT

A set of surgical instrument for use with expandable spinal implants. The set of instruments includes box chisels designed to prepare a site for implant placement. The set of instruments also includes inserter/expander instruments configured to place and expand an implant at the site. The set of instrument further includes assembling/collapsing instruments configured to assemble an implant, and collapse or reduce the height of an expanded implant.

### TECHNICAL FIELD

The present disclosure relates generally to the field of spinal surgery. In particular, the present disclosure relates to devices and instruments for use with spinal implants, and methods of using spinal implant devices and instruments.

### BACKGROUND

A wide variety of intervertebral implants and associated instruments have been utilized for stabilizing adjacent vertebral elements and facilitating the development of bone union between the vertebral elements. In some configurations, the intervertebral implants are adjustable. That is, the intervertebral implants are designed to expand from a first height to a second height. An example of an expandable intervertebral implant is disclosed in U.S. Pat. No. 5,489,307.

A number of devices and instruments are required to prepare for, handle, and operate expandable implants during a surgical procedure. In general, improvement has been sought with respect to such devices, instruments, and methods.

### SUMMARY

In one aspect, the present disclosure relates to a box chisel having a hollow structure. The hollow structure includes a first surface and a second opposite surface, each of the first and second surfaces having a cutting edge. The hollow structure also includes a rasping structure formed in one of the first and second surface.

In another aspect, the present disclosure relates to a chisel instrument having an elongated structure. The elongated structure includes a cutting edge and a first side facing in an opposite direction from a second side. The elongated structure also includes rasping structure formed in at least one of the first and second sides adjacent a distal end of the elongated structure.

In still another aspect, the present disclosure relates to a surgical instrument for expanding an expandable implant. The surgical instrument includes a sliding member and an instrument structure having an implant mounting arrangement. The mounting arrangement includes first and second portions adapted for insertion into the expandable implant. The sliding member is configured to slide relative to the instrument structure and expand the implant mounting arrangement such that the first and second portions expand the implant in an expansion direction.

In yet another aspect, the present disclosure relates to a surgical instrument for expanding an expandable implant. The surgical instrument includes a mounting structure configured for receipt of the expandable implant, a handle, and an actuator. The instrument is configured such that expansion of the mounting structure in a linear direction causes an expandable implant mounted on the mounting structure to expand from a non-expanded configuration to an expanded configuration.

In another aspect, the present disclosure relates to a surgical instrument for expanding an expandable implant. The surgical instrument includes a body defining a longitudinal axis extending between a proximal end and a distal end. The body defines a channel. The surgical instrument also includes a handle, an actuator configured to slide within the channel, and mounting structure having first and second members. When the actuator is slid toward the distal end of the body, the first and second members become spaced apart from one another.

In still another aspect, the present disclosure relates to a surgical instrument for expanding an expandable implant. The surgical instrument includes, an elongated housing, a rail member, and a channel defined by the elongated housing and the rail member. The surgical instrument also includes an actuator positionable within the channel, and configured to space apart the elongated housing and the rail member to thereby expand an expandable implant from a non-expanded configuration to an expanded configuration.

In yet another aspect, the present disclosure relates to a surgical instrument including a body, mounting structure located at a distal end of the body, a handle located at a proximal end of the body, and a moveable sleeve positioned between the handle and the mounting structure. The mounting structure is configured to insert within an opening of an implant and includes first and second member. When the sleeve moves toward the distal end of the body, the first and second members move toward one another.

In another aspect, the present disclosure relates to a surgical instrument including an engagement member having first and second cantilevers, and a sleeve positioned to slide along the engagement member. The sleeve is positionable at first and second positions. At the first position, the first and second cantilevers are spaced apart from one another a first distance. At the second position, the first and second cantilevers are spaced apart from one another a second distance, the second distance being less than the first distance.

In still another aspect, the present disclosure relates to a surgical instrument having a scissor arrangement. The scissor arrangement includes a first arm connected to a second arm at a pivot location. The surgical instrument also includes mounting structure located at a distal end of the scissor arrangement, the mounting structure being defined by shaped tips of the first and second arms. The mounting structure is configured to insert within an opening of an implant, and the shaped tips are configured to grasp structure formed within the opening of the implant. When the first and second arms, are pivoted, the shaped tips also pivot towards one another to cause a mounted implant to collapse from a first height to a reduced height.

In yet another aspect, the present disclosure relates to a kit. The kit includes at least one box chisel configured to prepare an implant site, an expandable implant, a first instrument configured to expand the expandable implant from a non-expanded configuration to an expanded configuration, and a second instrument having tips to engage the expandable implant to orient the expandable implant in a non-expanded configuration.

A variety of examples of desirable product features or methods are set forth in part in the description that follows, and in part will be apparent from the description, or may be learned by practicing various aspects of the disclosure. The aspects of the disclosure may relate to individual features as well as combinations of features. It is to be understood that both the foregoing general description and the following detailed description are explanatory only, and are not restrictive of the claimed invention.

### BRIEF DESCRIPTION OF THE DRAWINGS

FIG. 1 is a perspective view of a kit including instruments and an implant that can be used during a spinal surgery, in accord with the principles of the present disclosure;

FIG. 2 is an elevational view of the implant of FIG. 1 positioned between two vertebral elements, the implant being shown in a non-expanded configuration;

FIG. 3 is an elevational view of the implant of FIG. 1 positioned between two vertebral elements, the implant being shown in an expanded configuration;

FIG. 4 is an exploded perspective view of the implant shown in FIG. 1;

FIG. 5 is a front elevational view of the implant of FIG. 4;

FIG. 6 is a top perspective view of one embodiment of a box chisel instrument, shown in FIG. 1, in accord with the principles of the present disclosure;

FIG. 7 is a bottom perspective view of the box chisel instrument of FIG. 6;

FIG. 8 is a top plan view of the box chisel instrument of FIG. 6;

FIG. 9 is a front elevational view of the box chisel instrument of FIG. 8;

FIG. 10 is a partial, side, cross-section view of the box chisel instrument of FIG. 9;

FIG. 11 is a top perspective view of another embodiment of a box chisel instrument, shown in FIG. 1, in accord with the principles of the present disclosure;

FIG. 12 is a bottom perspective view of the box chisel instrument of FIG. 11;

FIG. 13 is a top plan view of the box chisel instrument of FIG. 11;

FIG. 14 is a front elevational view of the box chisel instrument of FIG. 13;

FIG. 15 is a perspective view of one embodiment of an inserter/expander instrument, shown in FIG. 1, in accord with the principles of the present disclosure;

FIG. 16 is a perspective view of the end of the inserter/expander instrument of FIG. 15;

FIG. 17 is an exploded assembly view of the inserter/expander instrument of FIG. 15;

FIG. 18 is a side elevational view of a housing of the inserter/expander instrument, and shown in FIG. 17;

FIG. 19 is a bottom plan view of the housing of FIG. 18;

FIG. 20 is a side elevational view of a rail of the inserter/expander instrument, and shown in FIG. 17;

FIG. 21 is a bottom plan view of the rail of FIG. 20;

FIG. 22 is a side elevational view of an actuator rod of the inserter/expander instrument, and shown in FIG. 17;

FIG. 23 is a side elevational view of the inserter/expander instrument of FIG. 15, shown with an implant mounted at an end;

FIG. 24 is a cross-sectional view of the inserter/expander instrument of FIG. 23, shown without the mounted implant and without an actuator rod;

FIG. 25 is a cross-sectional view of the inserter/expander instrument of FIG. 23, shown without the mounted implant and with an actuator rod;

FIG. 26 is an enlarged perspective view of the mounted implant of FIG. 23, the mounted implant being shown in a non-expanded configuration;

FIG. 27 is an enlarged perspective view of the mounted implant of FIG. 26, shown in an expanded configuration;

FIG. 28 is a schematic representation of another embodiment of an inserter/expander instrument, in accord with the principles of the present disclosure;

FIG. 29 is a schematic representation of still another embodiment of an inserter/expander instrument, in accord with the principles of the present disclosure;

FIG. 30 is a schematic representation of yet another embodiment of an inserter/expander instrument, in accord with the principles of the present disclosure;

FIG. 31 is a perspective view of one embodiment of an assembling/collapsing instrument, shown in FIG. 1, in accord with the principles of the present disclosure;

FIG. 32 is an alternative embodiment of the distal end of the assembling/collapsing instrument of FIG. 31;

FIG. 33 is an exploded assembly view of the assembling/collapsing instrument of FIG. 31;

FIG. 34 is a side elevational view of one embodiment of an engagement structure shown in FIG. 33;

FIG. 35 is a top plan view of the engagement structure shown in FIG. 34;

FIG. 36 is a side elevational view of the assembling/collapsing instrument of FIG. 33;

FIG. 37 is a cross-sectional view of the assembling/collapsing instrument of FIG. 36, taken along line 37-37;

FIG. 38 is a perspective view of another embodiment of an assembling/collapsing instrument, shown in FIG. 1, in accord with the principles of the present disclosure; and

FIG. 39. is a partial perspective view of the assembling/collapsing instrument of FIG. 38, shown with an implant mounted on the distal end.

### DETAILED DESCRIPTION

Reference will now be made in detail to various features of the present disclosure that are illustrated in the accompanying drawings. Wherever possible, the same reference numbers will be used throughout the drawings to refer to the same or like parts.

I. Kit Generally

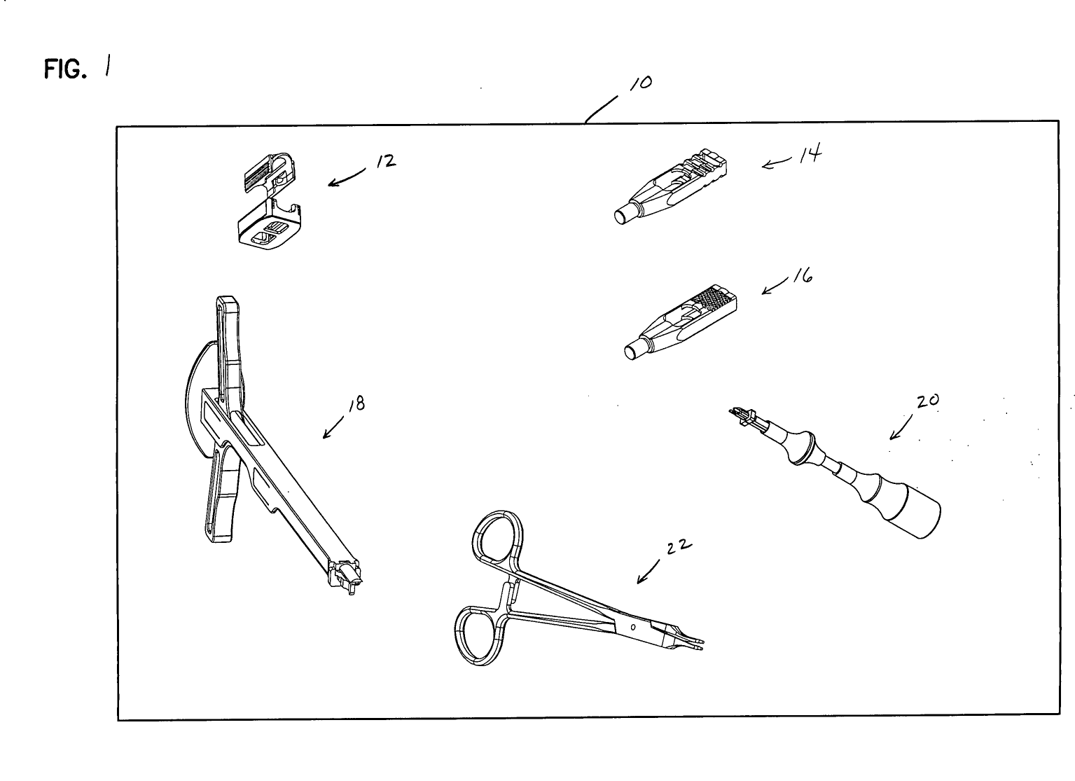

FIG. 1 illustrates a kit 10 (i.e., an instrument set) including a variety of instrument embodiments that can be used with an expandable implant during a spinal surgery procedure. The kit 10 is shown with one embodiment of an expandable spinal implant 12 for which the instruments are designed. It is to be understood that the illustrated implant is an example of one type of expandable implant, and that the instrument embodiments can be sized and configured for use with other expandable spinal implants having different sizes and structural configurations, in accord with the principles disclosed. Example implant embodiments for which the present disclosure of instruments are provided are described in U.S. Provisional Application No. 60/448,312 entitled EXPANDABLE INTERVERTEBRAL IMPLANT CAGE, filed Feb. 14, 2003, and its related application entitled EXPANDABLE INTERVERTEBRAL IMPLANT CAGE, having Ser. No. ______ Attorney Docket No. 6683.0069USU1, being filed concurrently herewith; both applications being incorporated herein by reference.

The illustrated kit 10 generally includes one or more embodiments of a box chisel (e.g. 14, 16), one embodiment of an inserter/expander (e.g. 18), and one or more embodiments of an implant assembling/collapsing instrument (e.g. 20, 22). Each of the instruments of the kit 10 is configured for use with a two-piece ratcheting-type implant, such as the expandable implant 12 shown.

II. Expandable Implant

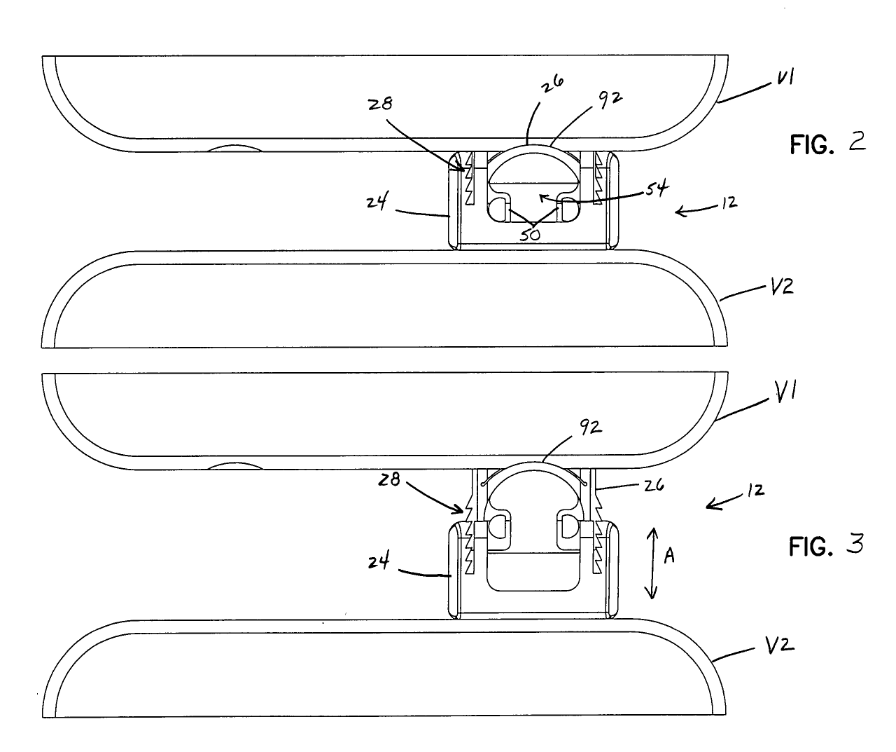

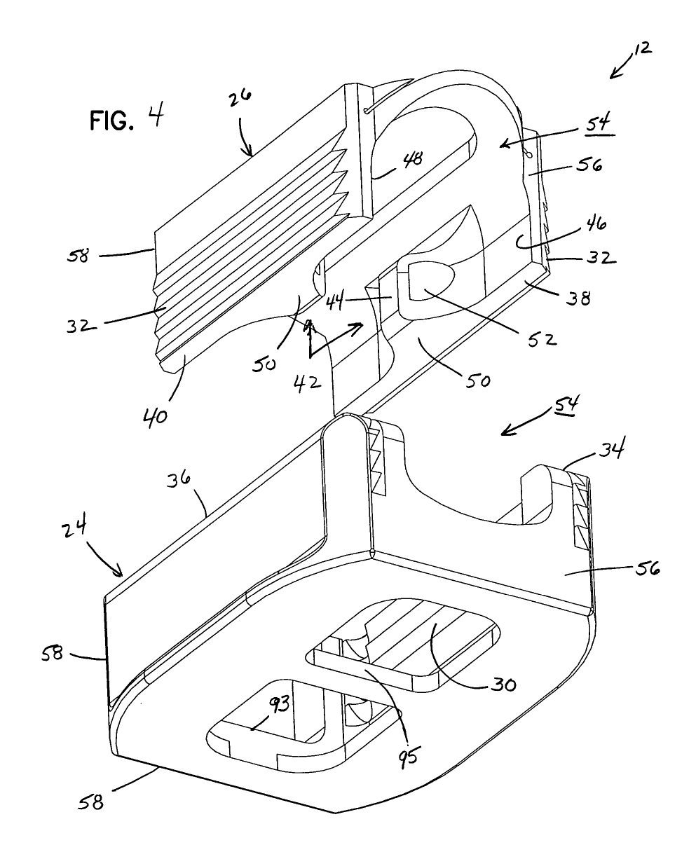

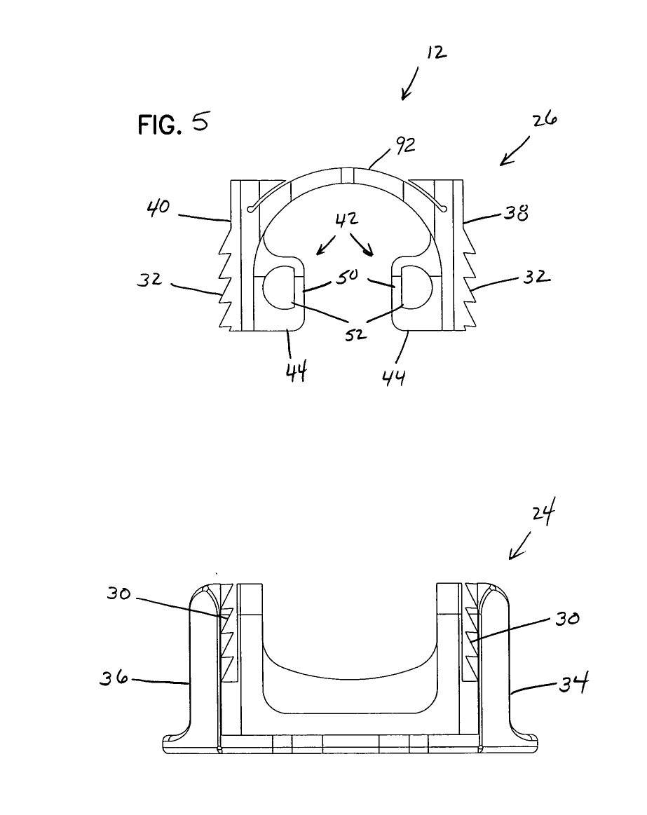

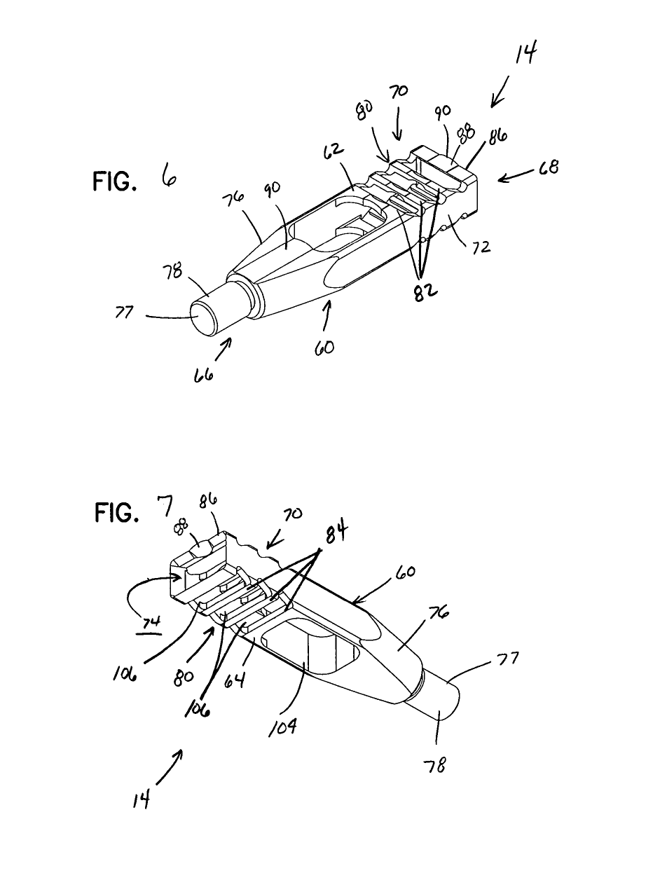

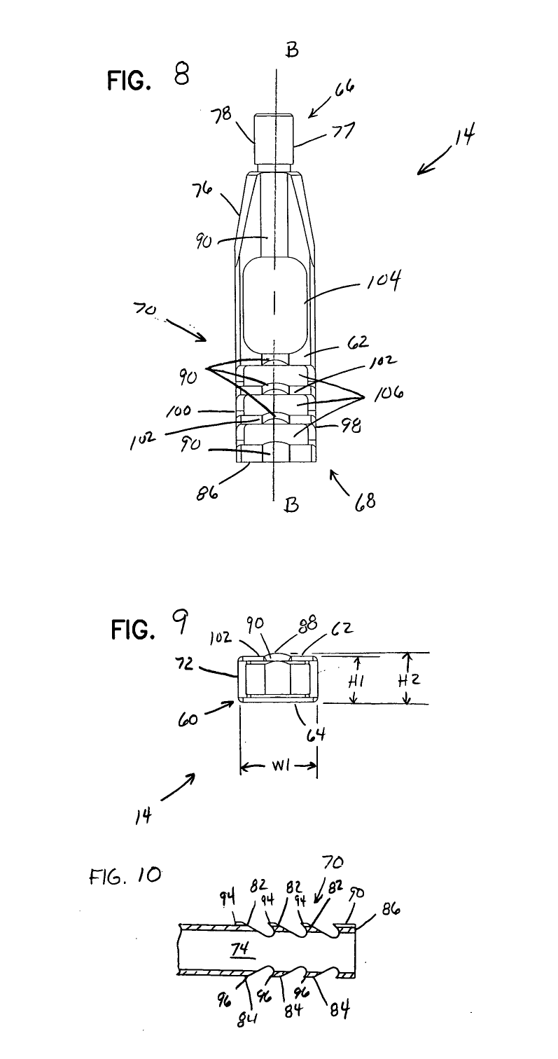

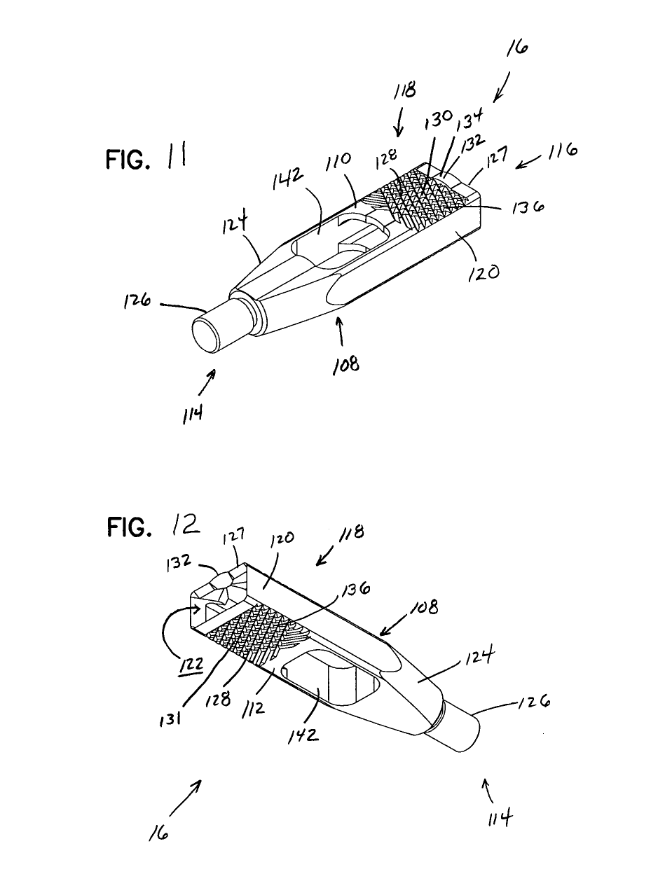

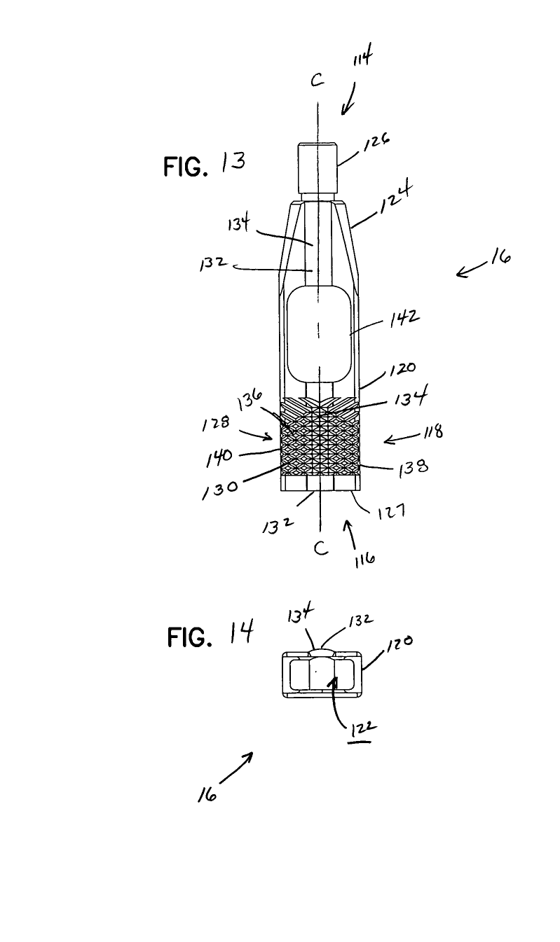

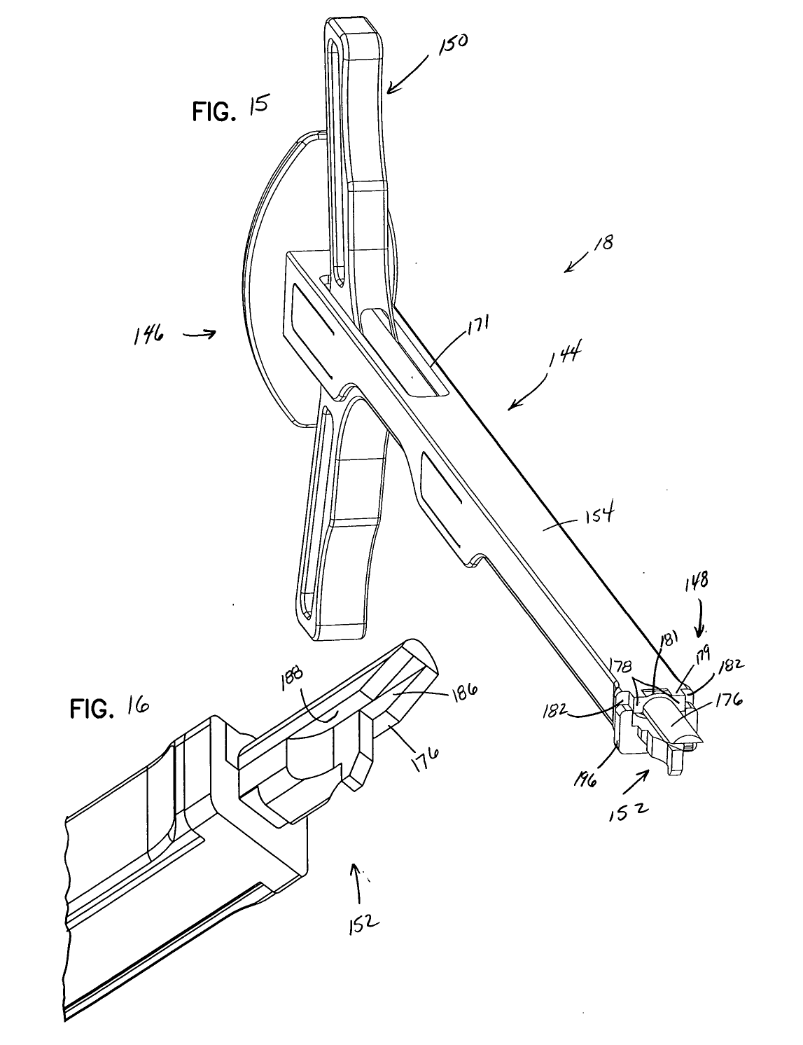

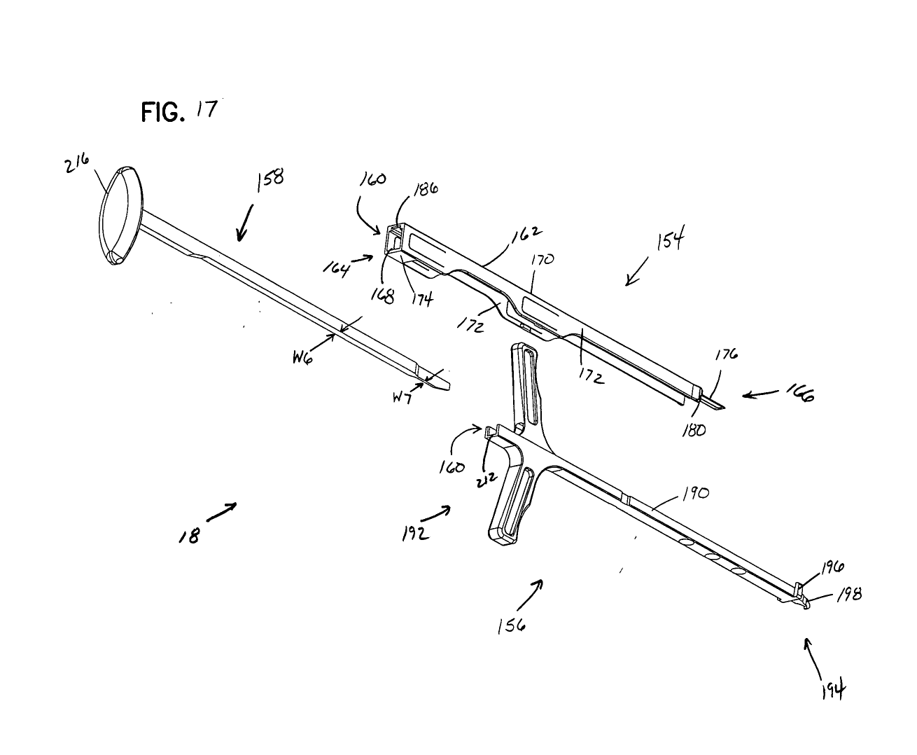

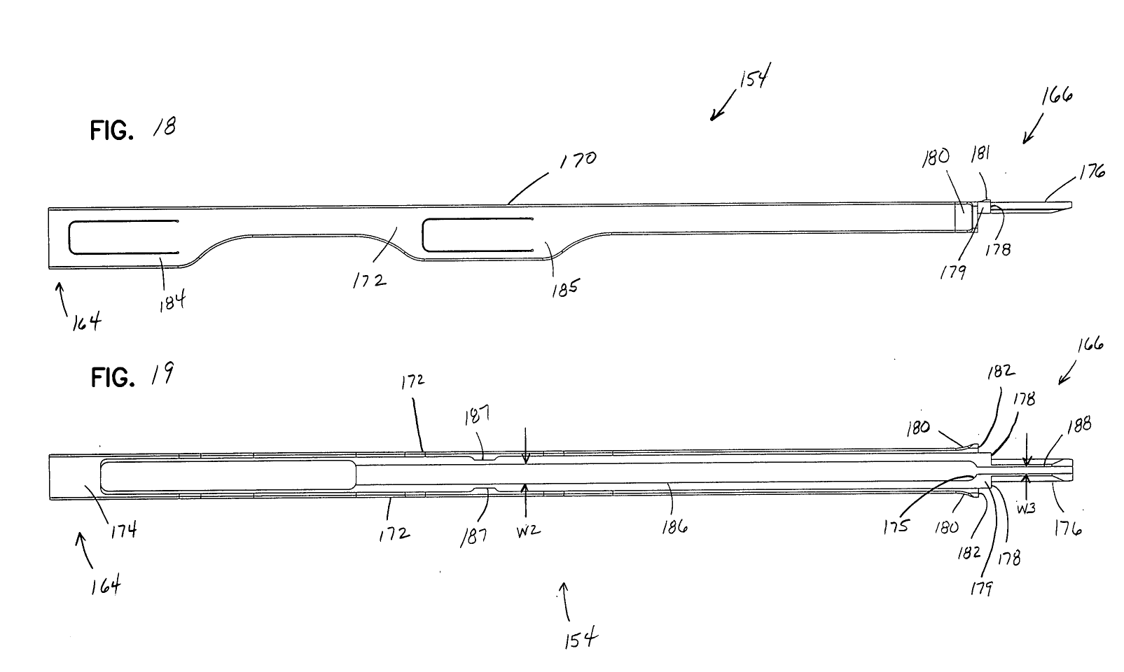

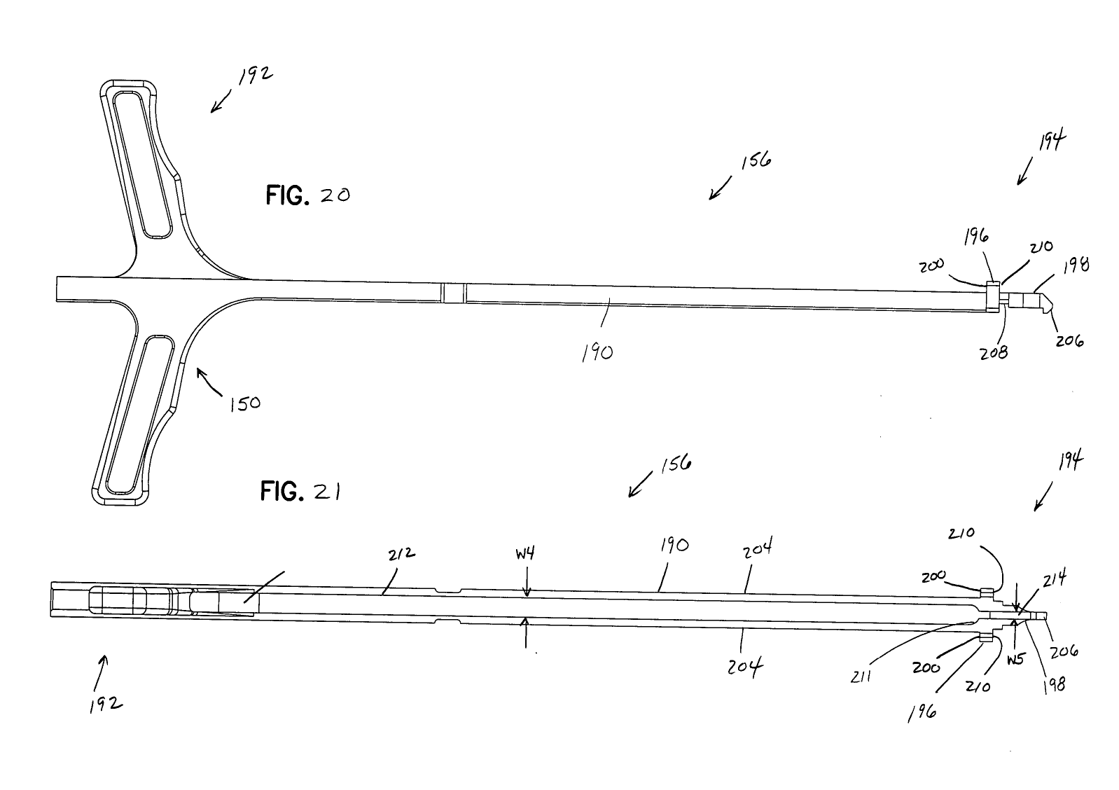

Referring now to FIGS. 2 and 3, the expandable implant 12 includes a first external member 24 and a second internal member 26. The implant 12 is configured for insertion between two vertebral elements V1, V2. Initially, the implant 12 is inserted in a non-expanded configuration (FIG. 2) and expanded to an expanded configuration (FIG. 3). Preferably the implant includes a locking arrangement 28 that permits expansion in a linear direction, as represented by arrow A in FIG. 3, and maintains the expanded configuration after the surgical procedure is complete. A linear direction is generally a non-rotational or non-angular direction, that is, a linear direction generally does not include expansion of one end pivoted about an opposite end. The implant 12 is also configured to permit retraction from the expanded configuration to the non-expanded configuration. The direction of expansion and retraction of the implant are of the same linear direction, as represented by arrow A.

Referring now to FIGS. 4 and 5, the locking arrangement of the implant 12 includes a first interlocking teeth structure 30 formed on first and second walls 34, 36 of the external member 24, and a corresponding second interlocking teeth structure 32 formed on first and second walls 38, 40 of the internal member 26. In use, the first and second interlocking teeth structures 30, 32 permit the implant 12 to incrementally expand or ratchet from the first non-expanded configuration to the second expanded configuration (FIGS. 2 and 3).

The illustrated implant 12 also includes an implant handling arrangement 42. Some of the surgical instruments of the kit 10 are designed to engage the implant handling arrangement 42 to handle and manipulate the implant 12 during a surgical procedure. The implant handling arrangement 42 of the illustrated embodiment is located on the internal member 26 and includes implant handling structures 44 formed on the inside walls surfaces 46, 48 of the first and second walls 38, 40. The handling structures 44 include a projection 50 defining a hole 52. The hole 52 is configured to receive an end of a surgical instrument.

When the internal and external members 24, 26 are assembled, openings 54 are formed at each of a first end 56 and second end 58 of the implant 12 (FIG. 4). As will be described in greater detail hereinafter, instruments can be inserted within the openings 54 to grasp or manipulate the implant 12 during a surgical procedure. In addition, the openings 54 provide access to an interior area of the implant. When the implant 12 has been inserted and expanded between two vertebral elements V1, V2 (FIG. 3), bone growth material can be packed within the interior area of the implant through either opening 54 of the first and second ends 56, 58.

III. Box Chisel

Prior to placement of an expandable implant at an implant site, the implant site between the two vertebral elements V1, V2 is first prepared. Preparation includes forming an implant site corresponding to the shape of the non-expanded implant. The box chisels 14, 16 shown in FIG. 1 can be used for preparing an implant site. The illustrated box chisels 14, 16 correspond to the configuration of the non-expanded implant 12 (FIG. 2).

Referring now to FIGS. 6-10, one of the box chisel embodiments 14 shown in the instrument kit 10 of FIG. 1 is illustrated. The box chisel 14 generally includes a structure 60 having a first substantially planar surface 62 and a second substantially planar surface 64. Generally, the distance between the first and second planar surfaces corresponds to the height of the implant in the non-expanded configuration.

Referring to FIG. 6, the box chisel 14 generally includes a proximal end 66 and a distal end 68 spaced along a longitudinal axis B-B (FIG. 8). A working portion 70 of the box chisel 14 is located adjacent to the distal end 68 of the box chisel. The working portion 70 is generally elongated and rectangular, and has a hollow construction 72 (FIGS. 6 and 9) extending from the distal end 68 of the box chisel 14 toward the proximal end 66. The hollow construction 72 defines a central open region or interior space 74 (FIGS. 7 and 10) extending along the longitudinal axis B-B of the box chisel 14.

A tapered region 76 is located adjacent to the proximal end 66 of the box chisel 14. Attachment structure 77 is located adjacent to the tapered region 76. The attachment structure 77 may include, for example, a threaded member 78 extending outward from the tapered region 76. A handle (not shown) can be attached to the threaded member 78 for manipulation and operation of the box chisel 14 during site preparation. In alternative embodiments, the box chisel 14 can include other types of attachment structure, such as a threaded hole formed in the tapered region, for attaching a handle.

Still referring to FIGS. 6 and 7, the box chisel 14 includes a perimeter cutting edge 86 and a surface texture or scraping arrangement 80. In use, the perimeter cutting edge 86 of the box chisel 14 is used as a leading cutting edge and can be forcibly inserted or tapped/hammered at a location between vertebral elements for site preparation. The perimeter cutting edge 86 cuts out a plug of material for removal. The perimeter cutting edge generally has a first height H1 and a width W1 (FIG. 9).

In the illustrated embodiment, the perimeter cutting edge 86 also includes an arcuate region 88. The acruate region extends upward from the first planar surface 62 and has a second height H2 greater than the first height H1. The arcuate region 88 is formed by a curvature 90 formed in the first planar surface 62. In the illustrated embodiment, the curvature 90 extends along the longitudinal axis B-B of the box chisel 14 (FIG. 8). The curvature 90 is configured to correspond to a curved portion 92 (FIG. 5) of the implant 12. Preferably, the cutting edge 86 and the scraping arrangement 80 are sized and shaped for preparing a channel or implant bore between adjacent vertebral elements V1, V2 corresponding to the particular profile of an expandable implant. As shown in FIGS. 2, 3 and 9, the curvature 90 formed along the first planar surface 62 of the box chisel 14 corresponds to the curved portion 92 of the implant 12. It will be appreciated, however, that the configuration of the perimeter cutting edge 86 can be square, circular, oval, etc., depending on the external configuration of the implant to be inserted between the vertebral elements V1, V2.

In general, the width W1, the first height H1, and the second height H2, of the box chisel 14 are sized to correspond to the size and configuration of the expandable implant 12, although the disclosed principles can be applied in a variety of sizes and applications. The width W1 of the box chisel 14 is generally defined between first and second sides 98, 100 of the box chisel, and is preferably between 0.375 and 0.750 inch; more preferably about 0.512 inches. The first height H1 of the box chisel 14 is generally defined between the first and second planar surfaces 62, 64, and is preferably between 0.250 and 0.500 inches; more preferably about 0.296 inches. The second height H2 of the box chisel 14 is generally defined between the second planar surface 64 and the curvature 134 formed in the first planar surface 62, and is preferably between 0.270 and 0.520 inches; more preferably about 0.316 inches.

To cut different sized channels, a set of chisels can be available for providing incrementally different sizes of cutting edges corresponding to a particular size implant. For example, chisels 14 having different widths W1 and heights H1, H2 can be made available to permit the surgeon to select a cutting edge corresponding to a particular implant configuration.

In the illustrated box chisel embodiment 14, the scraping arrangement 80 includes rasping structures or elements, such as teeth 82, 84 formed in each of the first and second planar surfaces 62, 64. The teeth 82, 84 of the box chisel 14 are designed to scrape the vertebral elements V1, V2 for further site preparation of the implant site.

The teeth 82, 84 of the scraping arrangement 80 illustrated in FIGS. 6-10 are configured for aggressive site preparation. That is, the teeth are configured to remove an amount of material from each of the vertebral elements V1, V2 for further sizing of the implant site. The teeth 82, 84 include cutting edges 94, 96 that extend upwardly from the first and second planar surfaces 62, 64 and are oriented such the cutting edges 94, 96 are directed or raked toward the proximal end 66 of the box chisel 14. It will be appreciated that the cutting edges 94, 96 of the teeth 82, 84 are parallel to the planar surfaces 62, 64 of the box chisel 14. In alternative embodiments, the cutting edges can be arranged to form a converging or diverging taper.

The teeth 82, 84 of the scraping arrangement 80 extend across each of the planar surfaces 62, 64 from the first side 98 of the box chisel 14 to the second side 100. The teeth 82 located on the first planar surface 62 also include curvatures 90 formed in a central portion 102 of each of the teeth (FIG. 8). The teeth 84 located on the second planar surface 64 are generally straight but may incorporate curvatures or shape structures corresponding to a different shaped implant.

As shown in FIG. 8, the scraping arrangement 80 includes three teeth located on each of the planar surfaces 62, 64. The three teeth are positioned between the first perimeter cutting edge 86 and the tapered region 76 of the box chisel. In an alternative embodiment, the scraping arrangement can have more or less than three teeth to facilitate preparations of the implant site.

Referring now to FIGS. 7 and 8, preferably the box chisel 14 includes openings or slots 106 located adjacent to each of the teeth 82, 84. The slots 106 are arranged in an alternating pattern with each of the teeth 82, 84. In the illustrated embodiment, the slots 106 extend across the planar surfaces 62, 64 from the first side 98 of the box chisel 14 to the second side 100. The slots 106 are oriented generally traverse to the longitudinal axis B-B of the box chisel, however, may be arranged in other orientations. Typically the slots 106 extend through the planar surfaces 62, 64 and into the interior space 74 of the box chisel. In use, material scraped from the vertebral elements V1, V2, by the teeth 82, 84 falls through the slots 106 into the interior space 74 of the box chisel 14 for removal.

The box chisel 14 also includes an aperture 104. The aperture 104 extends through the working portion 70 of the box chisel 14 and is in communication with the interior space 74 of the hollow construction 72. The aperture 104 is oriented generally transverse to the longitudinal axis B-B of the box chisel. That is, the aperture 104 extends from the first planar surface 62, through the working portion 70, to the second planar surface 64. The aperture is located between the last of the teeth 82, 84 of the scraping arrangement 80 and the tapered region 76 of the box chisel 14. In use, the aperture 104 provides an opening for removal of material scraped from the vertebral elements, and to facilitate cleaning between and at the rasping structures.

Referring now to FIGS. 11-14, another of the box chisel embodiments 16 shown in the instrument kit 10 of FIG. 1 is illustrated. The box chisel 16 generally includes a proximal end 114 and a distal end 116 spaced along a longitudinal axis C-C (FIG. 13). Similar to the previous embodiment, the box chisel 16 also includes a structure 108 having a first substantially planar surface 110 (FIG. 11) and a second substantially planar surface 112 (FIG. 12). A working portion 118 of the box chisel 16 is located adjacent to the distal end 116 of the box chisel. The working portion 118 is generally elongated and rectangular, and includes a hollow construction 120 defining an interior space 122.

The box chisel 16 also includes a width and first and second heights that are substantially the same as the previous box chisel embodiment 14. A tapered region 124 is located adjacent to the proximal end 114 of the box chisel 16. Attachment structure 126 is located adjacent to the tapered region 124 for attachment to a handle (not shown).

The box chisel 16 also has a perimeter cutting edge 127 and a surface texture or scraping arrangement 128. The scraping arrangement 128 includes rasping structure such as first and second roughening surfaces 130, 131 formed in each of the first and second planar surfaces 110, 112.

Similar to the previous embodiment, the roughening surface 130 also defines an arcuate region 132 that extends upward from the first planar surface 110. The arcuate region 132 is formed by a curvature 134 formed in the first planar surface 110 and extends along the longitudinal axis C-C of the box chisel 16. The curvature 134 is configured to correspond to the curved portion 92 of the implant 12.

The roughening surfaces 130, 131 in the illustrated embodiment each include a knurled or cross-hatched pattern construction 136. The cross-hatched pattern construction 136 functions to scrape the vertebral elements V1, V2 for further site preparation of the implant site. In particular, the roughening surfaces 130 of the scraping arrangement 128 are configured for detailed site preparation. That is, the cross-hatched pattern construction 136 is configured to rasp or roughen the surfaces of each of the vertebral elements V1, V2 to promote growth between the vertebral elements and the implant.

The cross-hatched pattern constructions 136 extend across each of the planar surfaces 110, 112 from a first side 138 of the box chisel 16 to a second side 140. Similar to the previous embodiment, the curvature 134 of the box chisel 16 extend through the cross-hatched pattern construction 136 located on the first planar surface 110.

Referring back to FIGS. 11 and 12, the box chisel 16 includes an aperture 142. The aperture extends through the working portion 118 of the box chisel 16 and is in communication with the hollow construction 120. The aperture 142 is oriented generally transverse to the longitudinal axis C-C of the box chisel. That is, the aperture 142 extends from the first planar surface 110, through the working portion 118, to the second planar surface 112. The aperture is located between the roughening surfaces 130, 131 and the tapered region 124 of the box chisel 16.

IV. Inserter/Expander

Once the implant site has been prepared, the expandable implant can be inserted between the two vertebrae V1, V2. FIGS. 15-27 illustrate one embodiment of the inserter/expander or placement instrument 18 shown in the instrument kit 10 of FIG. 1.

The placement instrument 18, as shown in FIGS. 15-16, includes a longitudinal body 144 having a first proximal end 146 and a second distal end 148. A handle 150 is located adjacent to the proximal end 146. Implant mounting structure 152 is located at the distal end 148.

As shown in FIG. 17, the placement instrument includes a housing 154, a rail 156, and an actuator rod 158. The body 144 of the placement instrument 18 is generally defined by the housing 154 and the rail 156. The actuator rod 158 is slidably located within a channel 160 of the body. The channel 160 is defined between the housing 154 and the rail 156.

Referring to FIGS. 17-19, the housing 154 generally includes a hollow construction 162 having a proximal end 164 and a distal end 166. An upper housing portion 170 and side portions 172 extend between the proximal and distal ends 164, 166. A lower housing portion 174 is located adjacent the proximal end 164. The upper and lower housing portions 170, 174 and the side portions 172 of the housing define a window or opening 168 at the proximal end 164. The opening 168 provides access to the channel 160 within the body 144 of the placement instrument 18.

Referring to FIG. 15, the upper housing portion 170 includes an opening 171. The opening is sized so that the handle 150 of the rail fits within the opening 171. As will be discussed in greater detail, the opening 171 is elongated to permit sliding movement of housing 154 relative to the rail 154 and handle 150 during use.

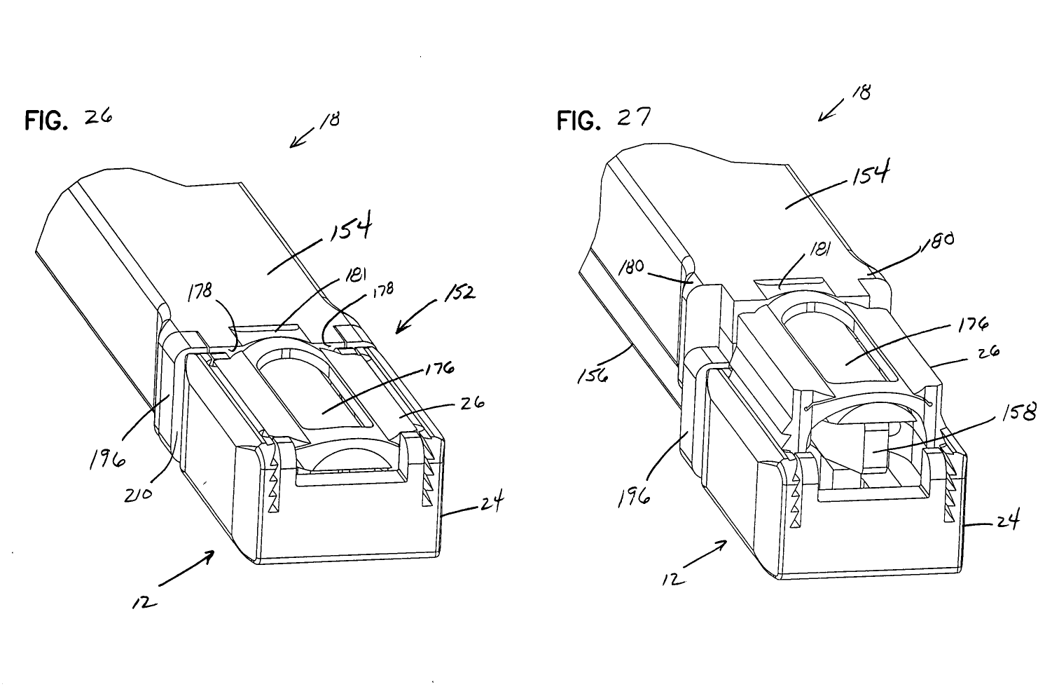

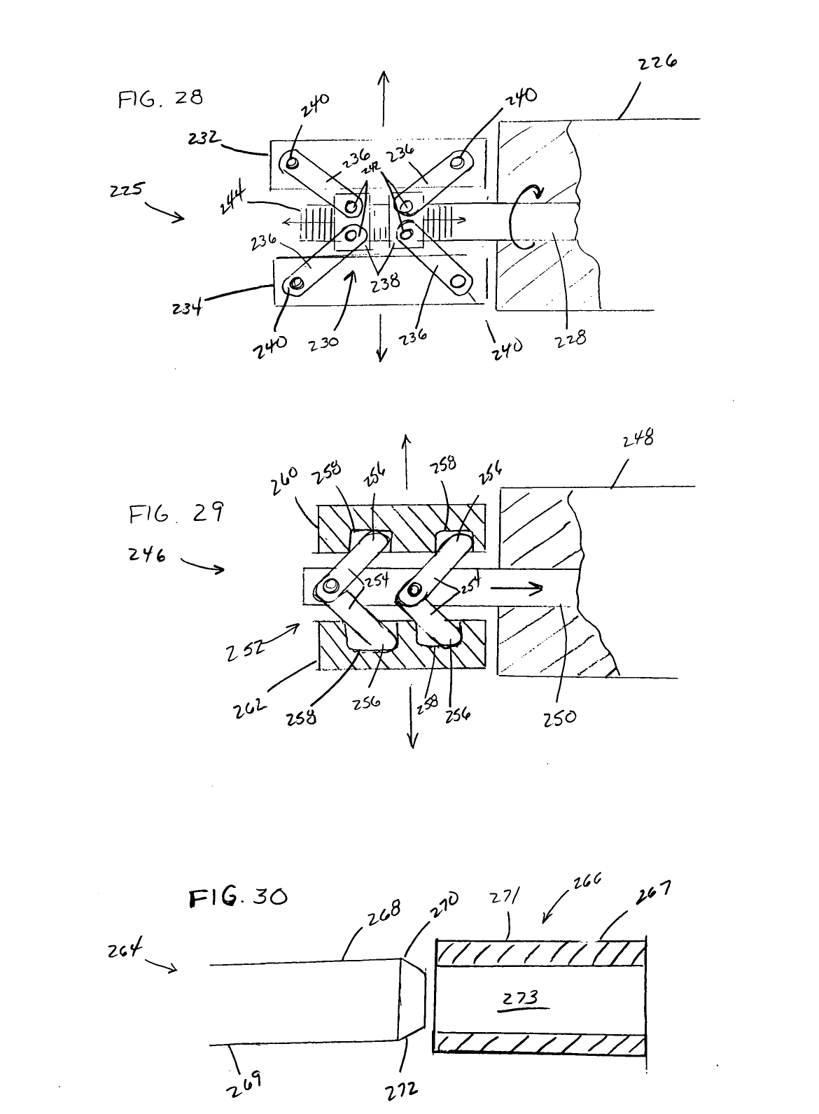

A longitudinal and axial projection 176 is located at the distal end 166 of the housing 154. In particular, the projection 176 extends outward from the upper housing portion 170 of the housing 154. The projection includes shoulders 178 formed at a base 179. In the illustrated embodiment, the base 179 includes an arcuate region 181 (FIGS. 15 and 18). The arcuate region 181 corresponds to the profile of the curved portion 92 of the expandable implant 12. The arcuate region 181 of the base 179 provides greater surface area to support the mounted implant 12 (FIG. 26) during the insertion and placement steps of the surgical procedure. In addition, the arcuate region 181 aids to more evenly distribute insertion forces on the end of the mounted implant during the surgical procedure.

In the illustrated embodiment, the distal end 166 of the housing 154 also includes a flared region 180. The flared region 180 has shoulder surfaces 182 located adjacent the base 179 of the projection 176. As will be discussed in greater detail, the shoulder surfaces 182 locate and maintain positioning of the rail 156 when the rail 156 and the housing 154 are assembled.

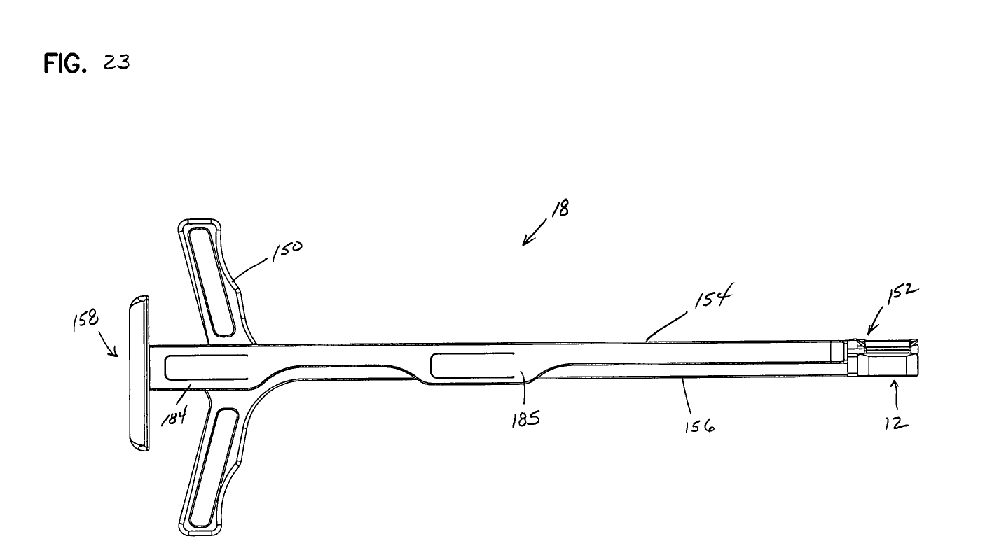

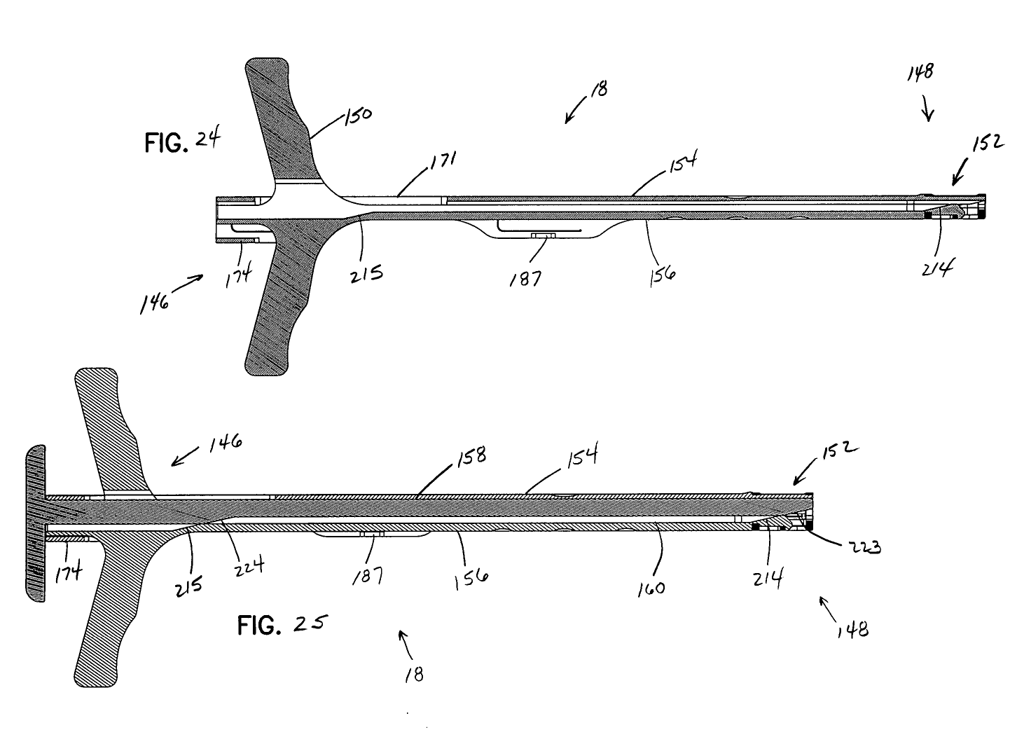

The sides 172 of the housing 154 include extended regions 184, 185. The extended regions generally define a maximum distance of separation allowed between the housing 154 and the rail 156 when the rail and housing are assembled. In particular, the proximal extended region 184 is interconnected to the lower housing portion 174. The intermediate extended region 185 includes tabs 187. The lower housing portion 174 and the tabs 187 hold the rail 156 in relation to the housing 154 during use (FIGS. 23 and 25).

Referring again to FIG. 17, the upper housing portion 170 includes a recess 186. The recess extends from the proximal end 164 to the distal end 166 (FIG. 19) and is configured to receive and guide the actuator rod 158 when the actuator rod is positioned within the channel 160. The recess 186 has a primary width W2 (FIG. 19) and a reduced width W3. The reduced width W3 is located adjacent to the distal end 166 of the housing 154 and extends into and along a bottom surface 188 of the projection 176.

Referring now to FIGS. 17, 20, and 21 the rail 156 includes an elongated member 190 extending between a proximal end 192 and a distal end 194. The handle 150 in the illustrated instrument is integral with the elongated member 190 and is located adjacent to the proximal end 192 of the rail 156.

A collar 196 and mounting tip 198 are located at the distal end 194 of the rail 156. The collar 196 includes proximal surfaces 200 extending upward along sides 204 of the rail. When assembled, the proximal surfaces 200 of the collar 196 abut the shoulder surfaces 182 of the housing 154 to locate and maintain the position of the rail 156 relative to the housing 154 (FIG. 15).

As shown in FIG. 20, the mounting tip 198 of the rail 156 has a hook 206. The hook 206 is configured to securely engage the expandable implant 12. In the illustrated embodiment, the hook 206 engages a catch or divider 95 (FIG. 4) formed in the external member 24 of the implant 12. In use, the hook 206 maintains the implant 12 on the mounting structure 152 of the inserter/expander 18 during insertion and placement steps of the surgical procedure.

In addition, the hook 206 can be used during a pull test. In particular, once the implant 12 is inserted and expanded at the implant site, the surgeon can pull the implant 12 by the hook 206 of the instrument 18 to ensure that the implant is securely positioned or placed.

Referring back to FIGS. 15 and 16, when the placement instrument 18 is assembled, the expandable implant is mounted on the mounting structure 152. The mounting structure 152 is generally defined by the projection 176 of the housing 154 and the mounting tip 198 of the rail 156. The second end 58 of the expandable implant 12 rests against the distal surfaces 210 of the collar 196 of the rail 156 and the shoulder 178 of the housing 154 (FIG. 26).

As shown in FIG. 20, the mounting tip 198 includes a notch 208. The notch 208 is located adjacent to the collar 196. The notch 208 is configured to receive a lower edge 93 (FIG. 4) of the second end 58 of the implant 12. When the edge 93 of the implant 12 is properly seated in the notch 208, the hook 206 clips onto or engages the catch 95 of the implant 12. The notch 208 ensures proper positioning of the implant 12 on the mounting structure 152 of the placement instrument 18.

Referring again to FIG. 17, a groove 212 is formed within the rail 156. The groove 212 extends from the proximal end 192 to the distal end 194 and is configured to receive and guide the actuator rod 158 when the actuator rod is positioned within the channel 160 of the assembled placement instrument 18. As shown in FIG. 21, the groove 212 has a primary width W4 and a reduced width W5. The reduced width W5 is located adjacent to the distal end 194 of the rail and extends into and along the mounting tip 198.

Referring now to FIGS. 21 and 24, the groove 212 includes a first ramped section 214 and a second ramped section 215. The first ramped section 214 is located at the mounting tip 198 of the rail 154. The second ramped section 215 is located toward the proximal end 192 of the rail 154. As will be described in greater detail, each of the ramped sections 214, 215 operates in cooperation with the actuator rod 158 to space the rail 156 and the housing 154 apart from one another, and in turn, expand the implant 12.

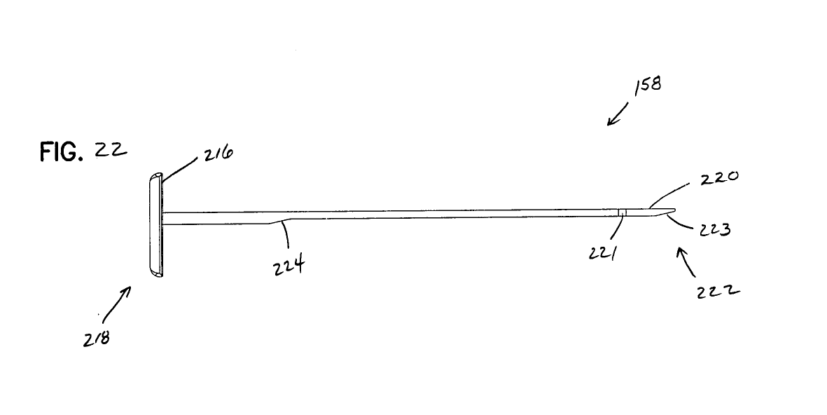

Referring now to FIG. 22, the illustrated actuator rod 158 of the present disclosure includes a head 216 located at a proximal end 218 and an actuator portion 220 located at a distal end 222. The head 216 is structured so that a surgeon can easily push the actuator rod 158 through the channel 160 (FIG. 25) with the palm of a hand to advance the actuator rod toward the mounting structure 152 of the placement instrument 18.

Referring back to FIG. 17, the rod 158 is constructed with a first width W6 and a second reduced width W7. The first width W6 is sized for sliding receipt within the channel 160 defined by the housing recess 186 and rail groove 212. The first width W6 of the actuator rod 158 corresponds to the primary widths W2, W4 of the recess 186 and groove 212. The second reduced width W7, located at the actuator portion 220 of the rod 158, is sized for sliding receipt within the correspondingly reduced widths W3, W5 of the recess 186 and groove 212.

In use, the varied widths of the placement instrument 18 components limit the depth at which the actuator rod 158 can be advanced within the channel 160. In particular, a tapered region 221 (FIG. 22) of the actuator rod 158 contacts a narrowing region 175 of the housing 154 (FIG. 19) and a narrowing region 211 of the rail 156 (FIG. 21) to stop forward movement of the actuator rod 158. This prevents the actuator rod from advancing past the distal end 148 of the placement instrument 18 and possibly causing unintended tissue, nerve or bone contact.

Referring again to FIG. 22, the actuator rod 158 includes a first inclined surface 223 and a second inclined surface 224. As can be understood by reference to FIG. 24, when the actuator rod is in a non-actuated position, i.e. not slid or advanced forward, the inclined surface 223 and 224 are located proximal to the ramped sections 214, 215 of the rail 154. As the rod slides forward toward the distal end 148 of the instrument 18, the inclined surfaces 223, 224 of the actuator rod 158 engage the ramped sections 214, 215 of the rail 156. As the actuator rod continues to advance, the rod 158 forces the housing 154 and the rail 156 apart; thereby linearly separating the projection 176 of the housing and the mounting tip 198 of the rail without any substantial pivoting between the housing and the rail. This in turn causes the implant to expand from the non-expanded configuration to the expanded configuration (FIGS. 26 and 27). As can be seen in FIGS. 24 and 25, the linear separation between the housing 154 and the rail 156 is limited. That is, the lower housing portion 174 and the tabs 187 maintain the relationship of the housing 154 and the rail.

In use, the placement instrument 18 is configured to place or insert the expandable implant 12 between the two vertebrae V1, V2. The installation procedure includes mounting the expandable implant 12 onto the mounting structure 152 of the placement instrument 18. At this point in the procedure, the external member 24 and the internal member 26 of the implant are assembled together and have a first non-expanded height (FIG. 26).

The implant 12 is inserted between two vertebral elements V1, V2 in the non-expanded configuration having a first non-expanded height (FIG. 2). The placement instrument 18 then expands the implant 12 by ratcheting the implant through a number of discrete incremental expansion positions to the expanded configuration. In particular, the actuator rod 158 is slid forward (FIG. 27) to separate the external member 24 and an internal member 26 of the implant 12 in the linear direction of expansion (represented by arrow A in FIG. 3). In some embodiment, markings (not shown) may be placed on the actuator rod 158 to indicate to the surgeon the height of expansion of the implant 12. After determining the desired expansion height for a patient, the surgeon can advance the actuator rod 158 to a particular marking that denotes the expanded implant height.

To remove the placement instrument 18 once the implant 12 is expanded, the actuator rod 158 is first removed from the channel 160. The housing 154 is then removed by sliding the housing 154, relative to the rail 156, out from the opening 54 of the implant. The opening 171 (FIG. 15) of the housing 154 accommodates the sliding motion of the housing relative to the handle 150. Last, the hook 206 of the rail 154 is disengaged from the catch 95 of the implant 12, and the rail 154 is then removed.

The placement instrument 18 provides several advantages. For example, the placement instrument is designed to easily insert the implant with minimal instrument adjustments. The placement instrument 18 also minimizes the detrimental effect on surrounding anatomy. In particular, the placement instrument 18 does not require interchanging multiple instruments to expand the implant from the non-expanded configuration to the expanded configuration. The actuator rod 158 is configured to easily slide and includes an enlarged palm area or head 216 to reduce stress on the surgeon's hand. The instrument 18 is also designed to fit within the opening 54 of the implant and does not extend beyond the profile of the implant; thereby the instrument 18 is less invasive and reduces trauma to the surrounding anatomy.

In addition, the double ramp/incline design of the instrument 18 (i.e. first and second ramped section 214, 215 of the rail 156 and first and second incline surfaces 223, 224 of the rod 158) maintains the parallelism of expansion between the housing 154 and the rail 156. This in turn, ensures that the implant 12 is equally and properly expanded at each end 56, 58. That is, the instrument 18 is designed so that both the proximal and distal ends 146, 148 of the instrument equally separate during operation. This design eliminates the possibility of unintended angular separation (i.e. the distal end 148 having a greater separation than the proximal end 146), which could cause the implant to ratchet or expand more at one end than the other.

Also, the placement instrument 18 includes three components. The components can be quickly assembled and disassembled for ease of use and cleaning.

Overall, the placement instrument 18 of FIGS. 15-27 includes a first member (e.g. the projection 176), a second member (e.g. the mounting tip 198), and an actuator. The actuator causes the first and second members to linearly move relative to one another in opposite directions to expand an implant. FIGS. 28-30 schematically represent other alternative embodiments having first and second members that linearly move in opposite directions for use in expanding an implant in accordance with the principles disclosed.

Specifically, FIG. 28 schematically illustrates an alternative inserter/expander embodiment 225 including a handle 226, an actuator 228, and an expanding linkage assembly 230. The expanding linkage assembly 230 in this embodiment 225 is interconnected to the actuator 228 and first and second members 232, 234. The first and second members 232, 234 may include fingers or other structure configured for receipt within the opening 54 of the implant 12 when in a non-expanded configuration. The first and second members 232, 234 may include mounting structure similar to the previous embodiment to retain an implant during insertion and placement.

The linkage assembly 230 of FIG. 28 includes link arms 236 and brackets 238. In the illustrated representation, four link arms 236 are provided. Other numbers of link arms can be used. Each of the link arms 236 includes a first end 240 pivotally coupled to one of the first or second members 232, 234, and a second end 242 pivotally coupled to one of the brackets 238.

The actuator 228 includes a threaded end 244. The threaded end 244 engages a threaded hole (not shown) in each of the brackets 238. As the actuator 228 is turned, the first and second members 232, 234 linearly separate or expand in opposite directions (represented by arrows). In particular, as a surgeon rotates or threads the actuator 228 into the brackets 238, the brackets axially move apart from one another (by means of a left-handed and a right-handed thread arrangement, for example). When the brackets 238 axially move apart, the link arms 236 rotate from a first angular orientation to a more vertical orientation; and in turn, drive the first and second members 232, 234 apart from one another. Separation of the first and second members 232, 234 accordingly expands a mounted implant.

To remove the insert/expander 225 once the implant has been expanded, the actuator 228 can be turned in an opposite direction to cause the brackets 238 to axially move toward one another, thereby collapsing the linkage assembly 230 and returning the first and second members 232, 234 to an initial position. The insert/expander 224 can then be removed from the opening 54 of the expanded implant 12.

Referring to FIG. 29, another inserter/expander embodiment 246 is shown. The inserter/expander 246 includes a handle 248, an actuator 250, and an expanding linkage assembly 252. The linkage assembly 252 includes link arms 254 pivotally connected to the actuator 250. Each of the link arms 254 includes a free end 256. Each of the free ends 256 is positioned within a slot 258 formed in first and second members 260, 262. The first and second members 260, 262 may include mounting structure similar to the previous embodiment to retain an implant during insertion and placement.

The first and second members 260, 262 are configured to linearly separate or expand in opposite directions (represented by arrows). In particular, as a surgeon axially pulls the actuator 250, the link arms 254 pivot and drive into the slots 258 to axially move the first and second members 260, 262 apart from one another. When the actuator 250 is axially pulled, the link arms 254 rotate from a first angular orientation to a more vertical orientation. Rotating the link arms 254 to a more vertical orientation drives the first and second members 260, 262 apart from one another, and accordingly, expands a mounted implant.

To remove the insert/expander 246 once the implant has been expanded, the actuator 250 can be pushed in the opposite direction to collapse the linkage assembly 252 and cause the first and second members 260, 262 to return to an initial position. The insert/expander 246 can then be removed from the opening 54 of the expanded implant 12.

Referring to FIG. 30, still another inserter/expander embodiment 264 is shown. In this embodiment, the inserter/expander 264 includes an expansion piece 266 and a wedge or driver 268 interconnected to a handle portion 269.

The wedge 268 has a conical or tapered region 270 at a first end 272. The expansion piece 266 in this embodiment may include a sheath 267 configured and sized for receipt within the opening 54 of the implant 12. The sheath 267 may include mounting structure similar to the previous embodiment to retain an implant during insertion and placement.

Preferably, the sheath 267 includes an expandable outer construction 271 defining an interior 273. When the sheath is positioned within the opening 54 of the implant, a user can slidably insert the wedge 268 into the interior 273 of the outer construction 271 to cause the sheath 267 to expand and thereby expand the mounted implant 12.

In general, the inserter/expander embodiments include a structure upon which an expandable implant is mounted. Each embodiment includes an actuator arranged to expand the structure in an expansion direction. The expansion direction in the illustrated inserter/expander embodiments is generally linear; however, the disclosed principles may be applied to provide expansion in a non-linear expansion direction. Expansion of the structure, in turn, expands the mounted implant from a non-expanded configuration to an expanded configuration. As can be understood, each of the embodiments is designed to structurally withstand expansion forces. In particular, the embodiments are structurally designed to created enough force to distract the disc space as well as expand the implant device.

V. Assembling/Collapsing Instrument

In use, the internal and external members 24, 26 of the implant 12 are assembled in relation to one another prior to insertion at an implant site. FIGS. 31-39 illustrate embodiments of the assembling/collapsing instruments shown in FIG. 1 that can be used to assemble the implant members 24, 26 in the non-expanded configuration.

In addition, the implant 12 is configured so that expansion from the non-expanded configuration to the expanded configuration is not permanent; that is, the surgeon can reduce the expansion height or can altogether collapse and remove the implant 12 after an installation and expansion procedure. The assembling/collapsing instruments of FIG. 31-39 are also configured to permit the surgeon to collapse the implant after the implant has been expanded.

In particular, the locking arrangement 28 (FIG. 4) of the implant can be disengaged by drawing or flexing the walls 38, 40 of the internal member 26 toward one another so that the second interlocking teeth structure 32 of the internal member 26 disengage from the first interlocking teeth structure 30 of the external member 24. To either assemble or collapse the implant, the assembling/collapsing instruments are configured to draw the walls 38, 40 of the internal member 26 toward one another to disengage the locking arrangement 28 of an implant 12.

Disengagement of the locking arrangement 28 is accomplished by inserting one of the assembling/collapsing instruments into the holes 52 of the implant handling structure 44, and flexing the walls 38, 40 together. When the first and second interlocking teeth structures 30, 32 have been disengaged, the external and internal members 24, 26 can be compressed to non-expanded configuration. The external and internal member 24, 26 may be compressed prior to installation (i.e. for assembly purposes) or while installed. In some situations, for example, a surgeon may determine after installation and expansion that the expanded height of the implant is too great. The surgeon can then disengage the interlocking teeth structures 30, 32 and permit the implant 12 to ratchet from the expanded configuration to a second, reduced expanded configuration.

Referring now to FIGS. 31-37, one embodiment of the assembling/collapsing instrument 20 of FIG. 1 is shown. The instrument 20 has a proximal end 276 and a distal end 278. A handle 274 is located at the proximal end 276. The instrument 20 also includes an engagement structure 284 including an implant handling arrangement 295. The implant handling arrangement 295 is positioned adjacent the distal end 278 of the instrument 20. A moveable sleeve 280 is positioned between the handle 274 and the implant handling arrangement 295.

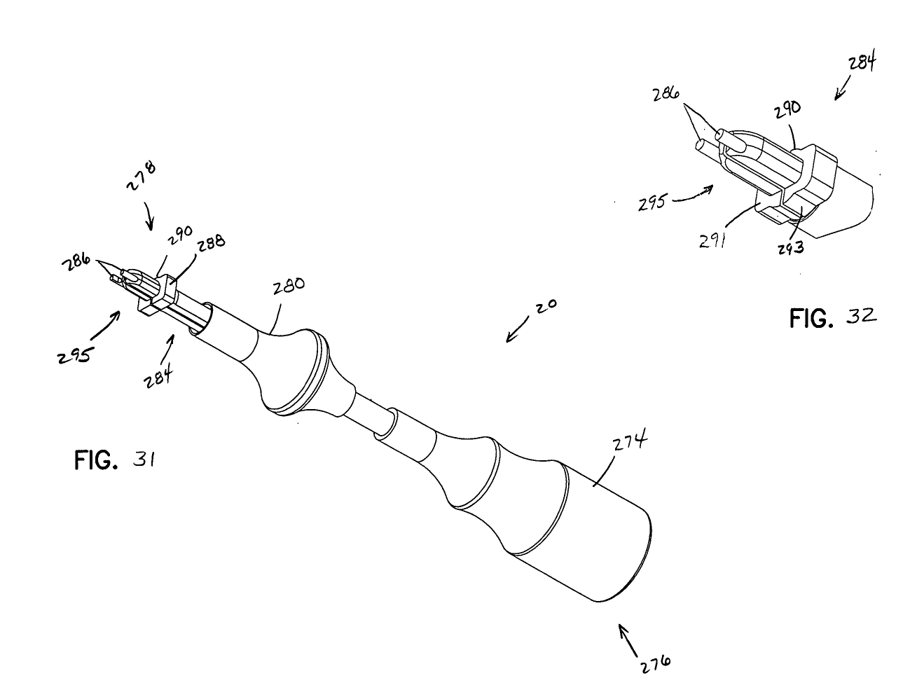

The engagement structure 284 of the instrument 20 includes projections 286 that engage the holes 52 of the implant handling structures 44 of the implant 12. In use, the engagement structure 284 is inserted within the opening 54 of the implant 12. A stop piece 288 is located a distance from the distal end 278 of the instrument 20. The distance is configured to limit insertion of the instrument 20 and provide an indication to the surgeon that the instrument 20 is fully engaged with the implant 12. The stop piece 288 of FIG. 31 includes an arcuate region 290 corresponding to the curved portion 92 of the implant 12. FIG. 32 illustrates an alternative stop piece embodiment 291 having a second arcuate region 293.

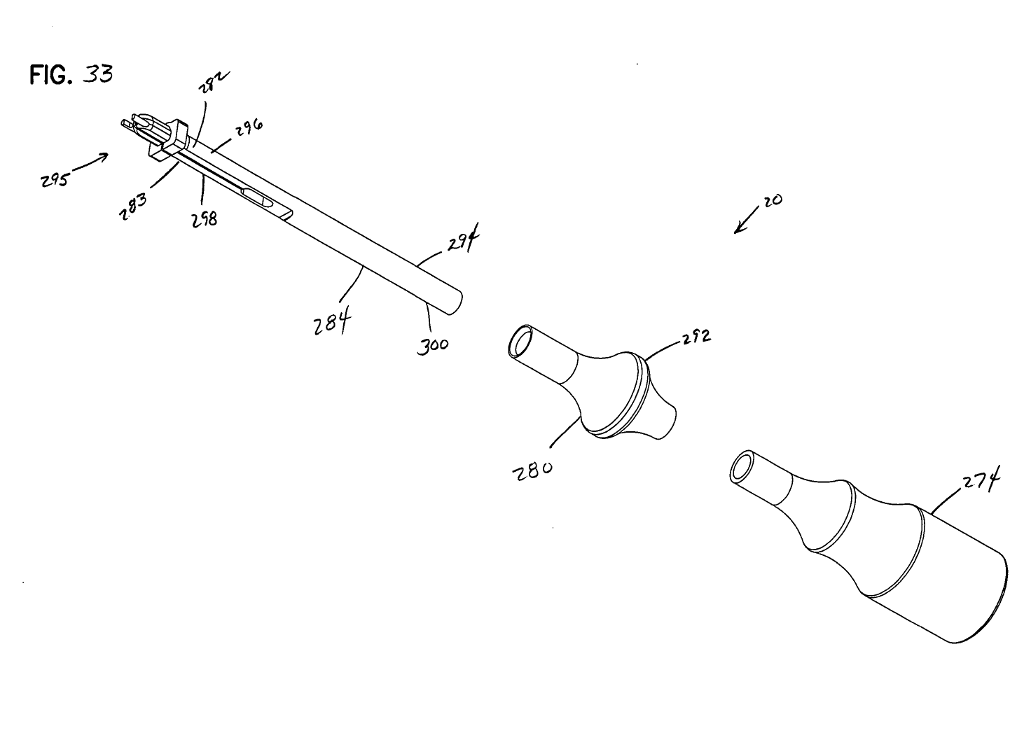

Referring now to FIG. 33, the engagement structure 284 of the instrument 20 includes an attachment portion 294 and first and second cantilever members 296, 298 extending from the attachment portion 294. Free ends 282, 283 of the first and second cantilever members 296, 298 define the handling structure 295. The attachment portion 294 in the illustrated embodiment is generally a dowel construction 300. The sleeve is 280 slidably positioned on the dowel construction 300, and the dowel construction is then connected to, e.g. threaded into, the handle 274.

The handle 274 in the illustrated embodiment is ergonomically designed for easy handling. Likewise, the moveable sleeve 280 is ergonomically designed, including a bulb-like region 292, for easy handling and sliding.

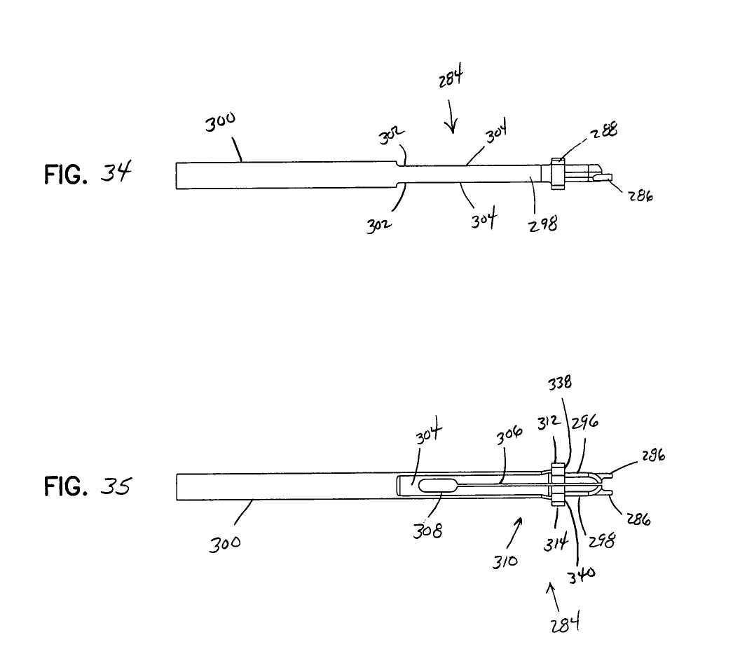

Referring now to FIGS. 34 and 35, the engagement structure 284 includes cut-away regions 302 that provide flats 304 on opposite sides of the structure 284. A center gap 306 and an aperture 308 are formed through the engagement structure between the flats 304. The center gap 306 defines each of the first and second cantilever members 296, 298.

The configuration of the aperture 308 and the center gap 306 provides a spring-type arrangement 310. In a relaxed state, the first and second cantilever members 296, 298 of the spring-type arrangement 310 are spaced apart from one another (FIG. 35). Preferably the spacing between the first and second cantilever member is such that the projections 286 extending outward from the members 296, 298 corresponds to the distance between the holes 52 of the implant handling arrangement 42 of the implant 12.

The stop piece 288 of the instrument 20 is defined by first and second stop sections 312, 314 formed on each of the first and second cantilever members 296, 298. The stop sections 312, 314 extend outward from the cantilever members. Shoulders 338, 340 of the stop section 312, 314 limit insertion of the instrument 20 in the implant 12. The shoulders 338, 340 prevent the distal end 278 of the instrument from extending through the implant and possibly causing unintended tissue, nerve, or bone contact.

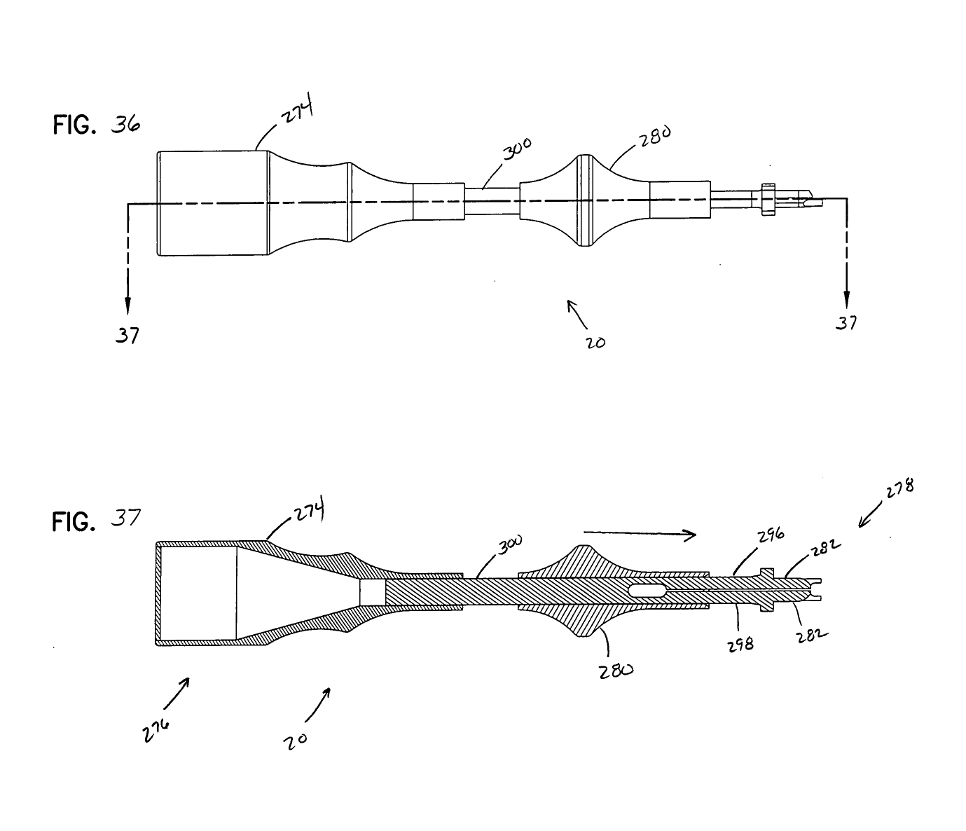

Referring now to FIGS. 36 and 37, in use, the instrument 20 is inserted into the holes 52 of an internal member 26 of the implant 12. The sleeve 280 is configured to slide forward toward the distal end 278 of the instrument 20. As the sleeve slides forward, the free ends 282 of the cantilever member 296, 298 are forced toward one another, in turn, drawing the walls 38, 40 of the internal member 26 of the implant (not shown) inward to disengage the locking arrangement 28. The implant 12 can then be collapsed for removal, or the height incrementally reduced as needed.

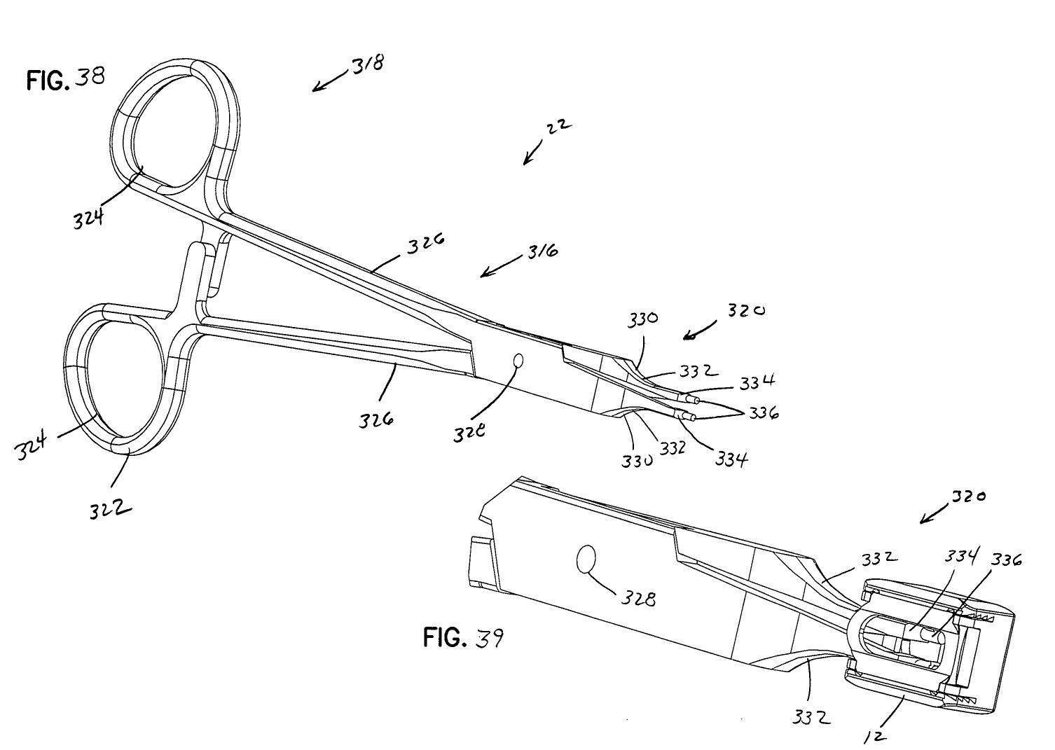

Referring to FIGS. 38 and 39, another embodiment of the assembling/collapsing instrument 22 of FIG. 1 is illustrated. In this embodiment, the instrument comprises a scissor-like arrangement 316 having a proximal end 318 and a distal end 320. The proximal end includes a handle 322, e.g. finger holes 324, for grasping and moving arms 326 of the arrangement 316 relative to one another. The arms are connected at a pivot point 328.

The distal end 320 includes shaped tips 330 sized for receipt within the holes 52 of the implant. The shaped tips 320 each include a necked region 332 leading to a shoulder 334, and a projection 336 extending outward from the shoulder 334. The necked region 332 is shaped so that the shaped tip 330 fits within the openings defined by the implant. The projections 336 are sized for receipt within the holes 52 of the implant 12. The shoulders 334 are configured to contact the handling structures 44 of the implant 12 when the shaped tips 330 are properly inserted into the holes 52 of the implant 12.

In use, a surgeon inserts the each of the shaped tip 330 into one of the holes 52 of the implant handling arrangement 42 of the implant. In this initial position, the shaped tips of the instrument 22 are spaced apart from one another. To draw the walls 38, 40 of the internal member 26 together, the surgeon squeezes the finger loops 324 to draw the shaped tips 330 inward, thereby disengaging the locking arrangement 28 of the implant 12. The implant 12 can then be collapsed for removal or the height reduced as needed.

The above specification provides a complete description of the INSTRUMENTS FOR USE WITH EXPANDABLE IMPLANTS, AND METHODS. Since many embodiments of the invention can be made without departing from the spirit and scope of the invention, certain aspects of the invention reside in the claims hereinafter appended.

1. A box chisel, comprising:
a) a hollow structure having a proximal end and a distal end, the hollow structure including:
i) a first side and a second opposite side, each of the first and second sides including a cutting edge located at the distal end of the hollow structure;
ii) at least a first rasping structure formed in one of the first and second sides.

2. The box chisel of claim 1, wherein the first rasping structure formed in the one of the first and second sides includes a plurality of teeth.

3. The box chisel of claim 2, wherein the one of the first and second sides further includes a plurality of slots extending through the hollow structure.

4. The box chisel of claim 3, wherein the slots are located in an alternating pattern between the plurality of teeth.

5. The box chisel of claim 1, wherein the first rasping structure formed in the one of the first and second sides includes a cross-hatched pattern construction.

6. The box chisel of claim 1, further including a second rasping structure formed in the other of the first and second sides.

7. The box chisel of claim 1, further including an arcuate region formed in one of the first and second sides.

8. A chisel instrument, comprising:
a) an elongated structure having a proximal end and a distal end, the elongated structure including:
i) a cutting edge located at the distal end of the elongated structure;
ii) a first side facing in an opposite direction from a second side; and
iii) rasping structure formed in at least one of the first and second sides adjacent the distal end of the elongated structure.

9. The chisel instrument of claim 8, wherein the elongated structure further includes a cross-section profile having a shaped region configured to match a non-planar implant profile, the shaped region being formed within at least one of the first and second sides of the elongated structure.

10. The chisel instrument of claim 9, wherein the shaped region includes a curvature extending along a length of the elongated structure.

11. The chisel instrument of claim 8, wherein the rasping structure includes a plurality of teeth formed in the one side.

12. The chisel instrument of claim 11 wherein the rasping structure further includes a plurality of slots extending through the one side, each of the slots being located in an alternating pattern between the plurality of teeth.

13. The chisel instrument of claim 11, wherein each of the slots is sized and configured such that material removed by the plurality of teeth pass through the slots into a central open region of the elongated structure.

14. The chisel instrument of claim 8, wherein the elongated structure includes rasping structure formed in each of the first and second sides.

15. The chisel instrument of claim 14, wherein each of the rasping structure formed in each of the first and second sides includes a plurality of teeth.

16. The chisel instrument of claim 8, wherein the rasping structure includes a roughened surface.

17. The chisel instrument of claim 16, wherein the roughened surface is a cross-hatched pattern construction formed on the one of the first and second sides.

18. The chisel instrument of claim 14, wherein each of the rasping structures formed in each of the first and second sides includes a roughened surface.

19. The chisel instrument of claim 9, wherein the rasping structure is formed across the shaped region of the elongated structure.

20. The chisel instrument of claim 19, wherein the rasping structure includes a plurality of teeth formed in the first side, the teeth defining a portion of the shaped region of the elongated structure.

21. The chisel instrument of claim 8, wherein the elongated structure includes a hollow construction.

22. A surgical instrument for expanding an expandable implant, the surgical instrument comprising:
a) an instrument structure having a implant mounting arrangement, the mounting arrangement including:
i) first and second portions adapted to be inserted into the expandable implant;
b) a sliding member, configured to slide relative to the instrument structure, the slide member being configured to expand the implant mounting arrangement such that the first and second portions expand a mounted expandable implant.

23. The surgical instrument of claim 22, wherein the first and second portions of the mounting arrangement are configured to move relative to one another in an expansion direction to expand the mounted expandable implant.

24. The surgical instrument of claim 23, wherein the expansion direction is a generally linear direction.

25. The surgical instrument of claim 22, wherein the sliding member includes a wedge configured to insert between the first and second portions of the implant mounting arrangement.

26. A surgical instrument for expanding an expandable implant, the surgical instrument comprising:
a) a mounting structure configured for receipt of the expandable implant;
b) a handle interconnected to the mounting structure;
c) an actuator configured to expand the mounting structure in a linear direction;
d) wherein expansion of the mounting structure in the linear direction causes an expandable implant mounted on the mounting structure to expand from a non-expanded configuration to an expanded configuration.

27. The surgical instrument of claim 26, wherein the mounting structure including a first member and a second member, the first and second members being moveable relative to one another in the linear direction.

28. The surgical instrument of claim 27, further including a linkage assembly interconnected to the first and second members and the actuator.

29. The surgical instrument of claim 28, wherein the actuator is configured to rotate relative to the handle, wherein rotating the actuator causes the first and second member to move relative to one another in the linear direction.

30. The surgical instrument of claim 28, wherein the actuator is configured to axially slide relative to the handle, wherein sliding the actuator causes the first and second member to move relative to one another in the linear direction.

31. The surgical instrument of claim 26, wherein the mounting structure includes a sheath, the sheath including an expandable outer construction defining an interior region, the interior region being configured for receipt of the actuator.

32. The surgical instrument of claim 31, wherein the actuator includes a wedge sized to expand the expandable outer construction of the sheath when inserted within the interior region of the sheath.

33. A surgical instrument for expanding an expandable implant, the surgical instrument comprising:
a) a body defining a longitudinal axis extending between a proximal end and a distal end, the body defining a channel;
b) mounting structure located at the distal end of the body, the mounting structure including a first member and a second member;
c) a handle located at the proximal end of the body;
d) an actuator configured to slide within the channel of the body;
e) wherein sliding the actuator toward the distal end of the body causes the first and second members to become spaced apart from one another.

34. A surgical instrument for expanding an expandable implant, the surgical instrument comprising:
a) an elongated housing;
b) a rail member interconnected to the elongated housing;
c) a channel defined by the elongated housing and the rail member;
d) an actuator positionable within the channel, the actuator being configured to space apart the elongated housing and the rail member to thereby expand the expandable implant from a non-expanded configuration to an expanded configuration.

35. The surgical instrument of claim 34, further including a handle located adjacent to the proximal end for operation of the surgical instrument.

36. The surgical instrument of claim 34, further including mounting structure defined by a distal end of the elongated housing and a distal end of the rail member, the mounting structure being configured for receipt of the expandable implant.

37. The surgical instrument of claim 36, wherein the mounting structure includes a projection extending outward from the distal end of the elongated housing.

38. The surgical instrument of claim 37, wherein the mounting structure includes a shaped tip extending outward from the distal end of the rail member.

39. The surgical instrument of claim 38, wherein the shaped tip includes a hook configured to engage and pull the implant after expansion.

40. The surgical instrument of claim 34, wherein the channel includes a ramped portion.

41. The surgical instrument of claim 40, wherein the actuator includes an inclined portion, the included portion configured to contact the ramped portion of the channel to spatially separate the housing from the rail member to expand the expandable implant.

42. The surgical instrument of claim 34, wherein the actuator includes a first inclined portion and a second inclined portion, the first inclined portion configured to contact a first ramped surface of the channel, the second inclined portion configured to contact a second ramped surface of the channel.

43. The surgical instrument of claim 42, wherein the first inclined portion is formed adjacent to a distal end of the actuator and the second inclined portion is formed adjacent to the proximal end of the actuator.

44. The surgical instrument of claim 43, wherein the first ramped surface of the channel is formed in the rail member adjacent to the distal end of the rail member.

45. The surgical instrument of claim 44, wherein the second ramped surface of the channel is formed in the rail member adjacent to the proximal end of the rail member.

46. The surgical instrument of claim 45, wherein the first and second inclined portions and the first and second ramped surfaces are positioned such that proximal and distal ends of the actuator rise simultaneously as the actuator slides along the channel in a first direction.

47. A surgical instrument, comprising:
a) a body defining a longitudinal axis extending between a proximal end and a distal end;
b) mounting structure located at the distal end of the body and configured to insert within an opening of an implant, the mounting structure including a first member and a second member;
c) a handle located at the proximal end of the body;
d) a moveable sleeve positioned between the handle and the mounting structure;
e) wherein moving the sleeve toward the distal end of the body causes the first and second members to move toward one another.

48. A surgical instrument, comprising:
a) an engagement member having a proximal end and a distal end, the engagement member including first and second cantilevers extending toward the distal end, the distal end being sized and configured for insertion into an opening of an implant;
b) a handle coupled to the proximal end of the engagement member;
c) a sleeve positioned to slide along the engagement member, the sleeve is positionable at:
i) a first position wherein the first and second cantilevers are spaced apart from one another a first distance, and
ii) a second position wherein the first and second cantilevers are spaced apart from one another a second distance, the second distance being less than the first distance.

49. The surgical instrument of claim 48, wherein the first and second cantilevers define handling structure configured to grasp an expandable implant

50. The surgical instrument of claim 49, wherein the handling structure includes projection extending outward from the distal end of the engagement structure, the projections being configured to grasp and maneuver the expandable implant.

51. A surgical instrument, comprising:
a) a scissor arrangement including a first arm connected to a second arm at a pivot location located between a proximal end and a distal end of the scissor arrangement;
b) mounting structure located at the distal end of the scissor arrangement, the mounting structure being defined by shaped tips of the first and second arms, the mounting structure being configured to insert within an opening of an implant, and the shaped tips being configured to grasp structure formed within the opening of the implant;
c) whereby pivoting the first and second arms, and the shaped tips towards one another causes a mounted implant to collapse from a first height to a reduced height.

52. A kit for use in a surgical implant procedure, the kit comprising:
a) at least a first chisel configured for implant site preparation;
b) an expandable implant;
c) a first instrument having a proximal end and a distal end, the distal end including implant mounting structure, the first instrument being configured to expand the expandable implant from a non-expanded configuration to an expanded configuration;
d) a second instrument having a proximal end and a distal end, the distal end including tips for engaging the expandable implant to orient the expandable implant in a non-expanded configuration.

53. The kit of claim 52, further including a second box chisel, the first box chisel being configured for more invasive implant site preparation, the second box chisel being configured for less invasive implant site preparation than the first box chisel.

54. The kit of claim 53, wherein the first box chisel includes teeth arranged to remove material from adjacent vertebral elements.

55. The kit of claim 53, wherein the second box chisel includes a roughened surface configured to scrape adjacent vertebral elements.

56. The kit of claim 52, wherein the first instrument includes a first member and a second member configured to selectively move in opposite directions to expand the expandable implant from the non-expanded orientation to the expanded orientation.

57. The kit of claim 52, wherein the tips of the second instrument are configured to engage a first member of the expandable implant to flex the first member relative to a second member of the expandable implant for orienting the expandable implant in the non-expanded configuration.

58. The kit of claim 57, wherein the second instrument is configured to flex the first member to disengage a locking arrangement of the expandable implant for collapsing the expandable implant from the expanded configuration to the non-expanded configuration.

59. The kit of claim 57, wherein the second instrument is configured to flex the first member to an orientation for assembling the first member relative to the second member in the non-expanded configuration.
